(19) United States
(12) Patent Application Publication
(10) Pub. No.: US 2019/0328403 A1
(43) Pub. Date: Oct. 31, 2019

(54) CRANIAL RECESS TOOL

(71) Applicant: Medtronic, Inc., Minneapolis, MN (US)

(72) Inventors: Ryan T. Bauer, Plymouth, MN (US); Phillip C. Falkner, Minneapolis, MN (US); Randy S. Roles, Elk River, MN (US); Victoria Holderby, Arlington, TX (US); Rebecca A Miron, Center City, MN (US); Greg J. Doyle, White Bear Lake, MN (US); S. Shane Dexter, Keller, TX (US)

(21) Appl. No.: 16/377,300

(22) Filed: Apr. 8, 2019

Related U.S. Application Data
(60) Provisional application No. 62/663,696, filed on Apr. 27, 2018.

(51) Int. Cl. A61B 17/16

(52) U.S. Cl. CPC A61B 17/1633; A61B 2090/0811; A61B 17/1695

(57) ABSTRACT

This disclosure is directed to systems and methods that facilitate the removal of tissue for implantation of an implantable medical device into bone or other substantially rigid tissue of a patient. An example system may include a shroud for a bone drill, the shroud including a housing extending along an axis and defining an inner surface around the axis. The inner surface may terminate at an edge of the housing and defining a cavity. The cavity may be configured to receive a drill bit having a shank and a primary cutting surface extending substantially parallel to the axis. The shroud also may include a collar coupled to the housing and configured to engage a distal portion of the bone drill.

This application claims the benefit of U.S. Provisional Application number 62/663,696, filed Apr. 27, 2018, which is incorporated herein by reference in its entirety.

### TECHNICAL FIELD

The disclosure relates to surgical devices, and more particularly to systems and methods that facilitate the removal of tissue for implantation of an implantable medical device.

### BACKGROUND

Certain types of implantable medical devices may be used to treat nervous system conditions such as pain, psychological, sleep, or movement disorders. Depending on the application for which they are implanted in a patient, an implantable medical device may include a variety of electrical and/or mechanical components to deliver a therapy to the patient. For example, an implantable medical device may include at least one elongate electrical lead operatively coupled to an implantable pulse generator device. The elongate electrical lead may include one or more electrodes that deliver from the generator device electrical stimulation therapy, such as deep brain stimulation or cortical stimulation. In some examples, the implantable medical device may be fixed or secured to a portion of the anatomy of a patient, such as the cranium.

### SUMMARY

This disclosure is directed to systems and methods that facilitate the removal of tissue for implantation of an implantable medical device into bone or other substantially rigid tissue of a patient. For example, a shroud may attach to a bone drill in order to prevent skin or other tissue from contacting a drill bit of the drill. The shroud may include a housing extending along an axis and configured to receive the drill bit having a primary cutting surface extending substantially parallel to the axis. The shroud may also include a collar coupled to the housing and configured to engage a distal portion of the bone drill. An operator (e.g., clinician, surgeon, physician's assistant, or the like) may insert the shroud and drill bit through an incision in the skin to a target site. The operator may then operate the bone drill to cause the bit to remove tissue from the target area, such as a portion of a cranium, to create a recess into which at least a portion of the medical device may be implanted. The shroud may enable insertion of the drill bit through the incision as opposed to removing a portion of the skin from the bone to expose the bone. Therefore, the smaller incision enabled by the shroud may reduce healing time, reduce risk of infection, or both, compared to other techniques.

In some examples, the disclosure describes a shroud for a bone drill, the shroud including a housing extending along an axis and defining an inner surface around the axis, the inner surface terminating at an edge of the housing and defining a cavity, where the cavity is configured to receive a drill bit having a shank and a primary cutting surface extending substantially parallel to the axis; and a collar coupled to the housing, the collar configured to engage a distal portion of the bone drill.

In some examples, the disclosure describes a system that includes a drill bit having a primary cutting surface extending substantially parallel to an axis and a shank; a shroud including a housing extending along the axis and defining an inner surface around the axis, the inner surface terminating at an edge of the housing and defining a cavity, where the cavity is configured to receive the drill bit; and a collar coupled to the housing, the collar configured to engage a distal portion of a bone drill.

In some examples, the disclosure describes a method that includes creating an incision in a scalp of a patient. The method also includes inserting a drill bit and a shroud through the incision in the scalp and external of a cranium of the patient, the drill bit and the shroud attached to a bone drill. The method also includes guiding the drill bit and the shroud under the scalp and to a target site, where the shroud includes a housing extending along an axis and defining an inner surface around the axis, the inner surface terminating at an edge of the housing and defining a cavity, where the housing is configured to receive a drill bit having a shank and a primary cutting surface extending substantially parallel to the axis; and a collar coupled to the housing, the collar configured to engage a distal portion of the bone drill. The method also includes removing, via operation of the bone drill, at least a portion of the cranium to define a recess.

The details of one or more examples are set forth in the accompanying drawings and the description below. Other features, objects, and advantages of the disclosure will be apparent from the description and drawings, and from the claims.

### BRIEF DESCRIPTION OF DRAWINGS

FIG. 1A is a conceptual diagram illustrating an example bone drill for creating a recess for a medical device implantable in a cranium of a patient.

FIG. 1B is a conceptual diagram illustrating an example medical device implanted in a cranium of a patient.

FIG. 1C is a picture illustrating three example templates that may be used to visualize a position and/or an orientation of a recess on a scalp of a patient.

FIG. 1D is a conceptual diagram illustrating seven example diagrams indicating incision positions on a scalp of a patient.

FIGS. 2A through 2C are conceptual diagrams illustrating an example bone drill and shroud to facilitate the removal of tissue for implantation of an implantable medical device at least partially within bone or other substantially rigid tissue.

FIG. 3 is a flow diagram illustrating an example technique of creating a recess in bone or substantially rigid tissue of a patient to facilitate the removal of tissue for implantation of an implantable medical device.

FIGS. 4A through 4F are conceptual diagrams illustrating various views of an example system set up to guide a tool in forming a cranial recess.

FIG. 5 is a conceptual diagram illustrating a perspective view of an example template assembly frame of the system set up to guide a tool in forming a cranial recess.

FIG. 6 is a conceptual diagram illustrating a perspective view of an example shroud of the system set up to guide a tool in forming a cranial recess.

FIGS. 7A through 7C are conceptual diagrams illustrating an example system for forming the cranial recess after an incision through the patient's scalp, to expose an outer surface of cranium has been made.

FIGS. 8A through 8C are conceptual diagrams illustrating a perspective view, an exploded view, and a side view, respectively, of an example system including a template assembly and a shroud attached to a tool to form an interface between the tool and the template assembly.

FIGS. 9A and 9B are conceptual diagrams illustrating a perspective view and a cross-section view, respectively, of an example system to guide a tool in forming a cranial recess.

FIG. 10 is a conceptual diagram illustrating a perspective view of an example sizer to check a size of a recess.

FIG. 11 is a conceptual diagram illustrating a perspective view of an example sizer to check a size of a recess.

FIG. 12 is a conceptual diagram illustrating a perspective view of an example IMD shaped to fit the contour of a recess.

### DETAIL_{E}D DESCRIPTION

This disclosure is directed to systems and techniques that facilitate the removal of tissue for implantation of an implantable medical device at least partially within bone or substantially rigid tissue of a patient. In order to treat certain diseases or disorders, a medical device may be implanted within a patient, and that implant location may be at least partially within bone or substantially rigid tissue of the patient. For example, an implantable medical device (IMD) operatively coupled to an elongated lead body, such as an electrical stimulation lead or a drug delivery catheter, may be implanted at least partially within a recess created in the cranium of the patient and under the skin of the patient.

In some examples, a system may facilitate the removal of tissue for implantation of an IMD by including a shroud for a bone drill or similar tool. Typically, a clinician may remove a portion of the scalp (e.g., create an incision and flap of skin that can fold backward) of the patient to expose the portion of the cranium at which the IMD may be implanted. Once the cranium is exposed, the clinician may use a bone drill or other device to remove some of the bone and create a recess within the cranium. However, removal of this amount of scalp may result in longer recovery time for healing and an increase in the risk of infection. As described herein, a shroud around a portion of a drill bit may allow the drill bit and shroud to be inserted through a smaller incision of the scalp and to the target site of the cranium. The shroud is then configured to protect surrounding skin and tissue from the operating drill bit within the shroud and facing the bone of the cranium.

The shroud may include a housing extending along an axis and configured to receive a drill bit having a primary cutting surface extending substantially parallel to the axis. The shroud may also include a collar coupled to the housing and configured to engage a distal portion of the bone drill. An operator may insert the shroud and the drill bit through an incision in the scalp to a target site on the cranium. Since the shroud is configured to protect surrounding tissue opposite the cranium from the drill bit, the drill bit can operate against the bone without damaging tissue on the other side of the shroud. The operator may operate the bone drill to remove cranial tissue from the target area to create a recess in which the IMD may be implanted. Creating the recess for the IMD remote from the incision and without exposing the target area of the cranium may reduce healing time, reduce risk of infection, or both.

In other examples, a system to facilitate the removal of tissue for implantation of an implantable medical device may include a system to guide a bone drill or similar tool in forming a recess in which the IMD may be implanted. The system may include a template assembly and a shroud. The template assembly may include supports configured for confronting engagement with the outer surface of the cranium or the scalp adjacent to a linear or C-shaped incision exposing the cranium. The template assembly may include an aperture that projects onto the outer surface of the cranium to define a shape of the recess to be formed in the cranium. The shroud may be configured to attach to the bone drill and include an aperture through which a drill bit may protrude. The template assembly may enable a sliding engagement interface between the template assembly and the shroud to enable an operator to form the recess in a repeatable fashion.

Although the systems and techniques are described herein with respect to implantation of a medical device at least partially within the cranium for delivering deep brain stimulation (DBS) therapy, these systems and techniques may be used at any anatomical location in which a medical device may benefit from being implanted at least partially within bone or other substantially rigid tissue. For example, therapies delivered from an IMD may include electrical stimulation therapy to various tissue sites to treat a variety of symptoms or conditions such as chronic pain, tremors, Parkinson's disease, epilepsy, urinary or fecal incontinence, sexual dysfunction, obesity, or gastroparesis. Electrical stimulation may be used in different therapeutic applications, such as spinal cord stimulation (SCS), pelvic stimulation, gastric stimulation, or peripheral nerve field stimulation (PNFS). In other examples, the implanted IMD may be configured to deliver a fluid containing a drug or other therapeutic substance.

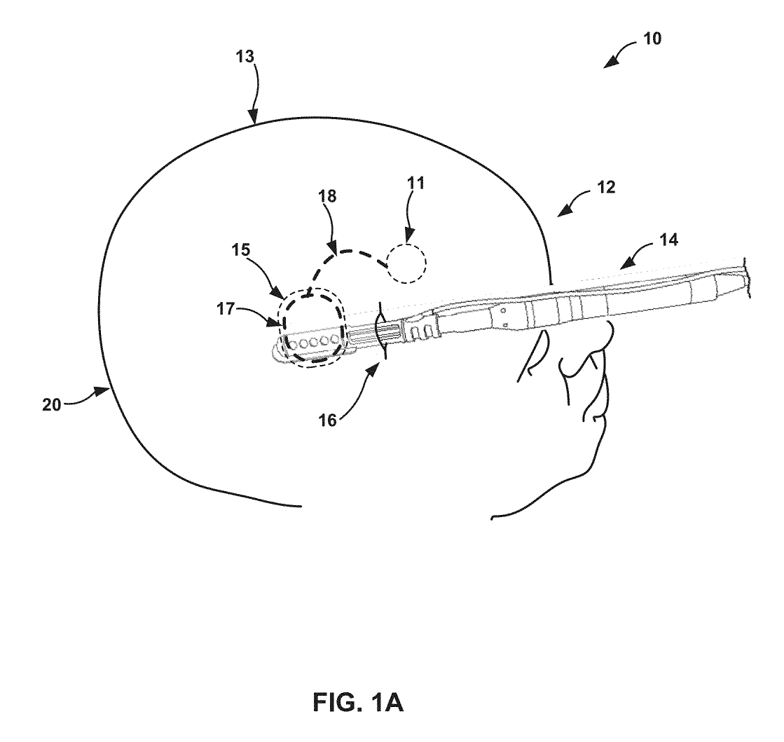

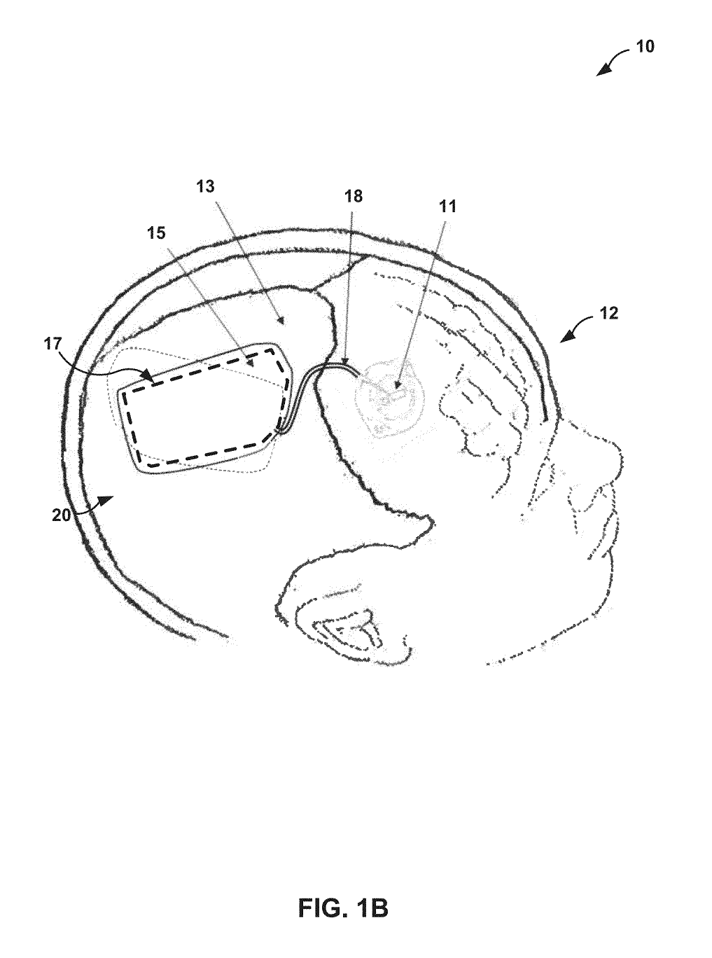

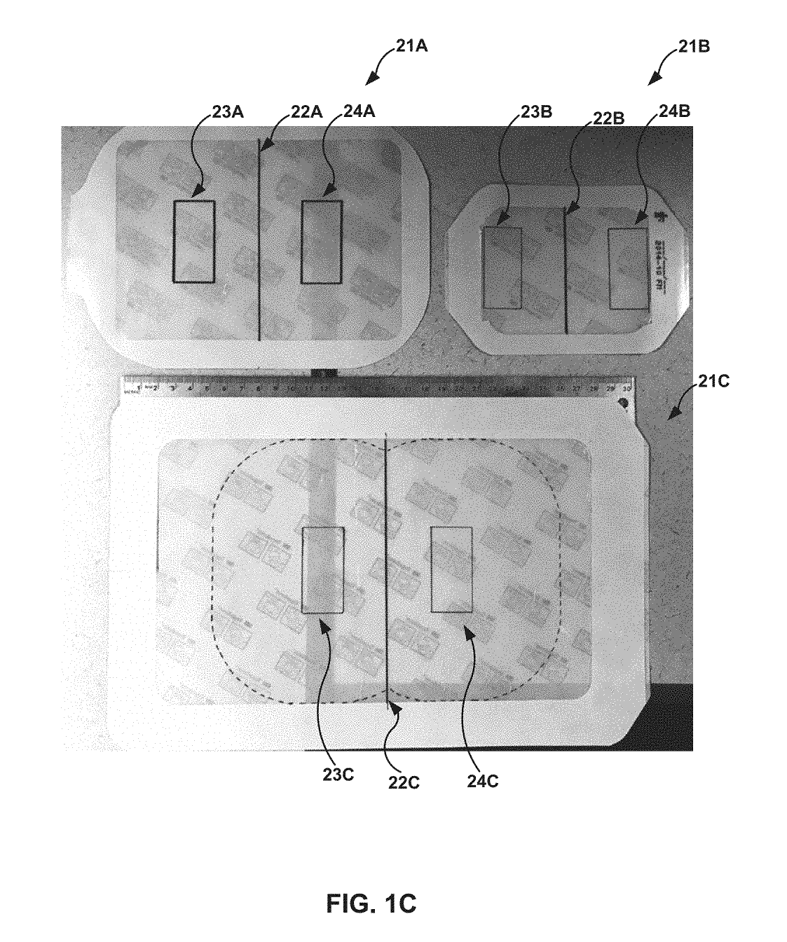

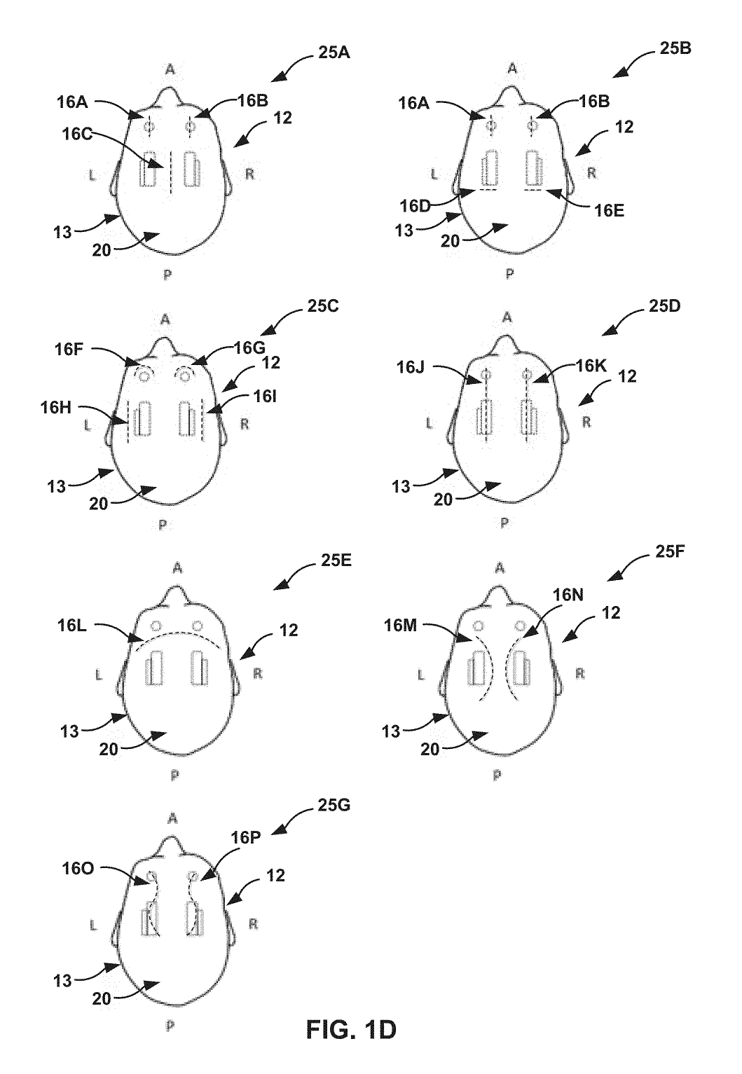

FIG. 1A is a conceptual diagram illustrating an example bone drill 14 that includes a shroud for creating recess 15 for a medical device 10 implantable in a cranium 13 of a patient 12, and FIG. 1B is a conceptual diagram illustrating an example medical device 10 implanted in cranium 13 of patient 12. Medical device 10 may include implantable medical device (IMD) 17 operatively coupled to a lead 18 (e.g., an elongate medical electrical lead). In some examples, lead 18 may include one or more electrodes disposed at a distal tip of lead 18 and/or at other positions at intermediate points along lead 18 that are implanted or otherwise placed adjacent to a target tissue. Electrodes of lead 18 may transfer electrical stimulation (e.g., as generated by IMD 17 to tissue of patient 12. The electrodes may be electrode pads on a paddle lead, circular (e.g., ring) electrodes surrounding the body of the lead, conformable electrodes, cuff electrodes, segmented electrodes (e.g., multiple electrodes located at the same axial location of the lead but different circumferential locations of the lead), or any other type of electrodes capable of forming unipolar, bipolar or multipolar electrode configurations for therapy. Using such electrodes of lead 18, IMD 17 may deliver electrical stimulation energy (e.g., current or voltage-based pulses) to the one or more targeted locations within patient 12 according to one or more therapy/stimulation program. In some examples, IMD 17 may deliver stimulation to the brain of patient 12 to provide DBS therapy or to stimulate the cortex of the brain. In some examples, IMD 17 may be used to treat any nervous system disorder including, but not limited to, epilepsy, pain, psychological disorders including mood and anxiety disorders, movement disorders (MVD), such as, but not limited to, dystonia, essential tremor, Parkinson's disease, and neurodegenerative disorders.

Although lead 18 is described as generally delivering or transmitting electrical stimulation signals, lead 18 may additionally or alternatively transmit electrical signals from patient 12 to IMD 17 for monitoring. Alternatively, or additionally, lead 18 and IMD 17 may be configured to provide other types of therapy through the delivery of a therapeutic agent to the target tissue of patient 12. For example, IMD 17 may additionally or alternatively deliver a fluid that includes therapeutic agent such as a pharmaceutical, biological, or genetic agent. In these examples, lead 18 may function as a catheter or IMD 17 may be otherwise coupled to a catheter.

As illustrated in FIG. 1A, bone drill 14 may be used to facilitate the removal of tissue for implantation of IMD 17. In some examples, a distal portion of bone drill 14 including a shroud and a drill bit may be configured to insert through incision 16 in scalp 20, external of cranium 13 of patient 12, to a target site under a portion of scalp 20. The target site may include, for example, a selected location in the bone of cranium at which recess 15 will be formed. As illustrated in FIG. 1A, an operator may insert the distal portion of bone drill 14, including a shroud and a drill bit, through incision 16 in the anterior to posterior direction (e.g., relative to patient 12). Anterior insertion of the distal portion of bone drill may improve operator control of bone drill, such as guiding bone drill 14 to the target location. In other examples, an operator may insert the distal portion of bone drill 14, including a shroud and a drill bit, through incision 16 in the medial direction, e.g., approximately normal to the sagittal plane. In other examples, the operator may insert bone drill 14 through incision 16 at any direction necessary to reach the target site for creation of recess 15. In some examples, bone drill 14 may be configured to remove at least a portion of cranium 13, e.g., bone tissue and/or substantially rigid tissue defining cranium 13, to define recess 15. Recess 15 may extend between approximately 1 millimeter and approximately 7 millimeters, such as between approximately 2 millimeters and approximately 4 millimeters, into cranium 13. Generally, the depth of recess 15 may be less than the thickness of cranium 13 such that a passage is not created through the entire thickness of cranium 13. However, in other examples, recess 15 may include a passage through cranium 13.

The drill bit may be shaped to form recess 15 that substantially corresponds to a shape of at least a portion of IMD 17. In some examples, IMD 17 may include, for example, an electrical pulse generator device. In some examples, medical device 10 may also include a cranial implant configured to secure lead 18 in cranial burr hole 11 such that electrodes of lead 14 remain disposed at one or more target sites within cranium 13. In some examples, lead 18 may be implanted to stimulate the nervous system of patient 12. In other examples, at least a portion of cranium 13 may be exposed to facilitate formation of recess 15. For example, “C-flap” incision, formed through scalp 20, may provide access to the cranium for the formation of burr hole 11, the implanting of lead 18, the formation of recess 15, and/or the implanting of IMD in recess 15. The systems and techniques of the disclosure may facilitate the forming of cranial recess 15, in a repeatable manner from patient to patient, so that a shape and depth thereof closely matches that of an IMD.

In some examples, a template may be used to visualize a position and/or an orientation of recess 15. FIG. 1C is a picture illustrating three example templates 21A, 21B, and 21C (collectively, “templates 21”) used to visualize a position and/or an orientation of recess 15 on scalp 20 of patient 12. Each respective template of templates 21 may include a respective midline 22A, 22B, and 22C (collectively, “midlines 22”), respective first recess locations 23A, 23B, and 23C (collectively, “first recess locations 23), and respective second recess locations 24A, 24B, and 24C (collectively, “second recess locations 24). In some examples, an operator may align a respective midline of midlines 22 with a midline of cranium 13 of patient 12 to position and/or orient a respective first recess location of first recess locations 23 and/or a respective second recess location of second recess locations 24. In some examples, an operator may use a marker or similar instrument to trace a respective first recess location of first recess locations 23 and/or a respective second recess location of second recess locations 24 on to scalp 20 of patient 12. Templates 21 may include any suitable material, such as, for example, polypropylene, polyethylene, combinations thereof, or the like. In some examples, templates 21 may include pliable material, such as, for example, surgical drape or similar material configured to conform with and/or removably adhered to a surface of scalp 20. In other examples, templates 21 may include rigid material, such as, for example, a material configured to enable an operator to trace, e.g., by a marker or similar instrument, the location of first and second recess locations 23 and 24 onto a surface of scalp 20. Using a respective template of templates 21 may improve visualization of a position and/or an orientation of recess 15, for example, when removing tissue with bone drill 14.

In some examples, incision 16 may include any suitable position, any suitable orientation, and any suitable shape on scalp 20 of cranium 13 of patient 12. FIG. 1D is a conceptual diagram illustrating five example diagrams 25A, 25B, 25C, 25D, 25E, 25F, and 25G (collectively, “diagrams 25) indicating possible incision positions on a scalp 20 of a cranium 13 of a patient 12. Each respective diagram of diagrams 25 includes a craniocaudal view (including labels of the anterior direction “A”, the posterior direction “P”, the left (sinister) side “L”, and the right (dexter) side “R”) of the position and orientation of incisions 16A through 16P (collectively, “incisions 16”) in scalp 20. For example, diagram 25A illustrates incisions 16A and 16B near the locations of two boreholes (not labeled for clarity) and incision 16C near the medial line between the locations of two recesses (not labeled for clarity). Diagram 25B illustrates incisions 16A and 16B near the locations of two boreholes and incisions 16D and 16E posterior to the locations of two recesses. Diagram 25C illustrates incisions 16F and 16G anterior to the locations of two boreholes and incisions 16H and 16I sinister and dexter to the respective locations of two recesses. Diagram 25D illustrates incisions 16J and 16K extending in the anterior-posterior direction between the locations of two boreholes the locations of two recesses. Diagram 25E illustrates incision 16L posterior to the locations of two boreholes and anterior to the locations of two recesses. Diagram 25F illustrates incisions 16M and 16N medial to the locations of two boreholes and the locations of two recesses. Diagram 25G illustrates serpentine shaped incisions 16O and 16P extending in the anterior-posterior direction between the locations of two boreholes the locations of two recesses. One or more example incisions 16 may be used in any suitable combination and other positions and orientations of incisions 16 are contemplated.

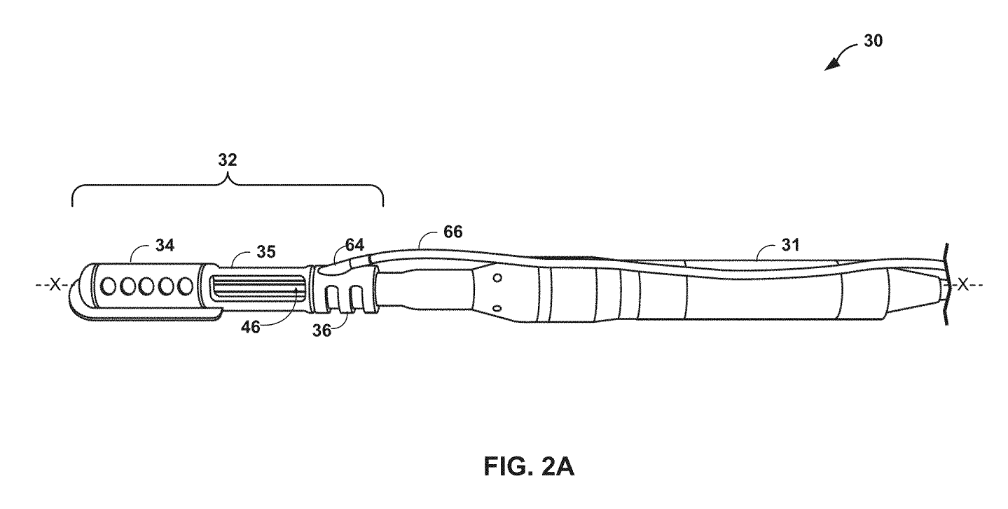

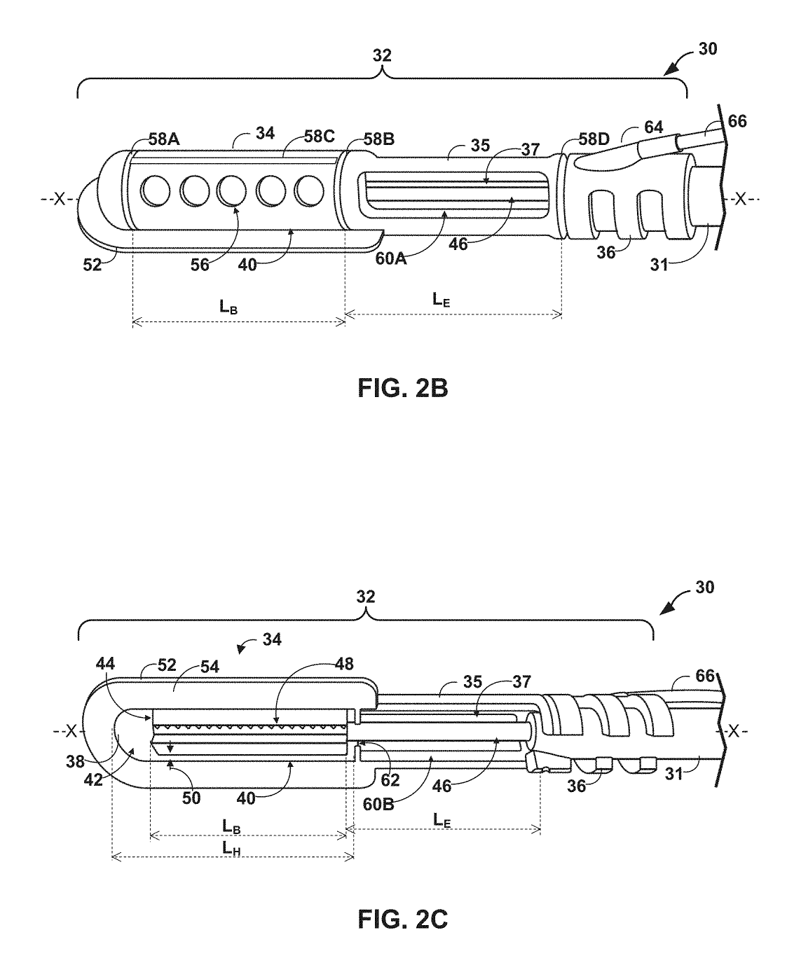

FIGS. 2A through 2C are conceptual diagrams illustrating an example bone drill 30 and shroud 32 to facilitate the removal of tissue for implantation of an implantable medical device at least partially within bone or other substantially rigid tissue. Bone drill 30 may be similar to bone drill 14 of FIG. 1A. As shown in the example of FIGS. 2A-2C, bone drill 30 includes drill housing 31 and is attached to shroud 32. Drill housing 31 may enclose a motor that is operatively coupled to a drive shaft. For example, the motor may operate to rotate the drive shaft. In some examples, bone drill 30 may include one or more attachments, including, for example, the drive shaft, operatively coupled to the motor. In some examples, the drive shaft may include one or more gears, for example, to change a rotational ratio from the motor and/or form an angle, such as a 90° angle, in a portion of the drive shaft. A distal portion of the drive shaft includes a coupling (e.g., a drill chuck) to engage a portion of a drill bit (e.g., shank 46). A portion of shroud 32 may be shaped or formed to engage a distal portion of drill housing 31.

Shroud 32 may include a housing 34 and a collar 36. In some examples, shroud 32 may include an extension member 35 coupling housing 34 to collar 36. Housing 34, extension member 35, and collar 36 may extend along an axis (e.g., the X-X axis), which may run parallel to the longitudinal axis of drill housing 31. Shroud 32 may be constructed of any suitable material, such as, for example, one or more of medical-grade metal (e.g., stainless steel, titanium, titanium alloy, or the like), medical-grade polymers (e.g., polyolefins, polyvinylchloride, methacrylates, polyethers, polyurethanes, polycarbonates, acetals, or the like), ceramic materials, or any combination thereof. In some examples, each of housing 34, extension member 35, and collar 36 may include distinct components joined by, for example, crimping, welding, mechanical fasteners or fastening systems, or the like. In other examples, housing 34, extension member 35, and collar 36 may be integrally formed as a single component. Shroud 32 may be formed by any suitable means, such as, for example, additive manufacturing, casting, machining, or molding.

As illustrated in FIG. 2C, housing 34 extends from a proximal portion to a distal portion and defines inner surface 38 around the X-X axis and defining cavity 42. Inner surface 38 may define any suitable shape, such as a curvilinear shape, a rectilinear shape, an irregular shape, or a combination thereof. For example, as illustrated in FIG. 2C, inner surface 38 may define a semi-cylindrical shape. In some examples, the shape of inner surface 38 may be selected to direct debris, such as bone or tissue fragments, and/or irrigation fluid out of housing 34. In some examples, inner surface 38 terminates at an edge 40 of housing 34.

Housing 34 defines a cavity 42. Cavity 42 is configured to receive a drill bit 44 having a shank 46 and a primary cutting surface 48, each cutting surface extending substantially parallel to the X-X axis and rotatable about the X-X axis. For example, cavity 42 may be shaped to allow an operator to insert shank 46 into a coupling on a distal portion of the drive shaft of bone drill 30 when collar 36 is engaged with a distal portion of drill housing 31. Additionally, or alternatively, cavity 42 may be shaped to allow an operator to position shroud 32 over drill bit 44 to engage collar 36 with a distal portion of drill housing 31 when shank 46 is engaged with a distal portion of the drive shaft of bone drill 30. In some examples, cavity 42 provide a predetermined clearance 50 between inner surface 38 and drill bit 44, e.g., primary cutting surface 48. In some examples, predetermined clearance 50 between inner surface 38 and drill bit 44 may be determined based on an average bone fragment size or upper bound of bone fragment sizes produced by drill bit 44 when operating in contact with bone or substantially rigid tissue. In this way, predetermined clearance 50 between inner surface 38 and drill bit 44 within cavity 42 may reduce friction between debris (e.g., removed bone or substantially rigid tissue) and drill bit 44 and/or inner surface 38, allow for debris to be removed from cavity 42 (e.g., by contact force from drill bit 44 against the bone debris and/or irrigation fluid), or both.

In some examples, one or more portions of shroud 32 may be configured to contact an outer surface of a cranium of a patient (e.g., cranium 13 of patient 12). For example, edge 40 may include a substantially smooth surface to enable a sliding engagement interface between the outer surface of cranium 13 and edge 40. In some examples, shroud 32 includes a flange 52 extending radially outward (e.g., relative to the X-X axis) from edge 40 of housing 34. Flange 52 may be integrally formed with housing 34 or secured (e.g. bonded, friction fit, or the like) to shroud 32 at or near edge 40. Flange 52 may include a substantially smooth surface 54 to enable a sliding engagement interface between the outer surface of cranium 13 and flange 52. In some examples, shroud 32 may include other components configured to contact an outer surface of cranium 13, such as, for example, rails, pedestals, or the like positioned at a proximal end of shroud 32 near collar 36. By contacting an outer surface of cranium 13, one or more portion of shroud 32, such as flange 52, may improve handling or stability of bone drill 30 as bone drill 30 is operated to form recess 15.

In some examples, housing 34 is configured to enable primary cutting surface 48 of drill bit 44 to radially extend between approximately 1 millimeter and approximately 7 millimeters, such as between approximately 2 millimeters and approximately 4 millimeters, beyond edge 40 and/or flange 52. For example, edge 40 and/or flange 52 may be disposed relative to the X-X axis such that primary cutting surface 48 of drill bit 44 may extend beyond a plane defined by edge 40 and/or flange 52 a predetermined amount. By extending beyond the plane defined by edge 40 and/or flange 52 a predetermined amount, drill bit 44 may, when operated, remove bone or substantially rigid tissue at a depth substantially equal to (e.g., equal to or nearly equal to) the predetermined amount. In some examples, the amount of primary cutting surface 48 extending beyond a plane defined by edge 40 and/or flange 52 may be adjustable. For example, housing 34 may be configured to rotate about an ellipse relative the X-X axis such that a first position of rotation enables a first amount of primary cutting surface 48 extending beyond a plane defined by edge 40 and/or flange 52, and a second position of rotation enables a second amount of primary cutting surface 48 extending beyond a plane defined by edge 40. An adjustable shroud may enable an operator to adjust the depth of recess 15, for example, to make multiple passes to achieve a desired depth of recess 15.

Housing may be any suitable length to receive drill bit 44, as discussed above. In some examples, drill bit 44 length “L_{B}” may be selected to be proportional to a length or a width of a desired recess (e.g., recess 15). In some examples, drill bit 44 may be equal to a length or a width of recess 15 to enable recess 15 to be formed in a single pass of drill bit 44. Housing 34 length “L_{H}” a predetermined amount greater than “L_{B}” to enable drill bit 44 to be coupled to drill housing 31, provide a predetermined clearance 50 at the distal end and/or proximal end of housing 34, or both. In some examples, length L_{H} of housing 34 may be between approximately 2 centimeters and approximately 6 centimeters, such as between approximately 3 centimeters and approximately 5 centimeters.

In some examples, housing 34 may define one or more apertures 56 (shown in FIG. 2B) through a portion of inner surface 38. Apertures 56 may be any suitable shape, such as circular or rectilinear. Apertures 56 may be configured to enable debris to exit housing 34. For example, force applied by operation of drill bit 44 to bone may expel debris through apertures 56. In some examples, cavity 42 may be irrigated with irrigation fluid to improve expulsion of debris through apertures 56 and/or cool tissue. By enabling expulsion of debris, apertures 56 may reduce friction between debris and drill bit 44 and/or inner surface 38. In some examples, apertures 56 also may reduce the weight of shroud 32. In some examples, apertures 56 also may enable an operator to confirm operation (e.g., rotation) of drill bit 44.

In examples in which shroud 32 includes extension member 35, extension member 35 may surround at least a portion of shank 46. In some examples, extension member may define a cavity 37. By surrounding at least a portion of shank 46, extension member 35 may reduce the risk of contact of tissue, e.g., scalp 20, with rotating components, such as shank 46. In some examples, extension member 35 may include one or more apertures 60A and 60B. In some examples, aperture 60A may reduce build-up of debris in extension member 35 to reduce friction between debris and rotating components, such as shank 46. In some examples, aperture 60B may enable extension member 35 to fit over shank 46 when engaging collar 36 with drill housing 31. A length “L_{E}” of extension member 35 in the axial direction (e.g., along the X-X axis) may be approximately equal to length “L_{H}” of housing 34 in the axial direction or length “L_{B}” of drill bit 44 in the axial direction. In this way, extension member 35 may provide a visual indication of the position of drill bit 44 under scalp 20 relative to incision 16.

Shroud 32 may include other indicators configured to provide at least one of a tactile indication or a visual indication of a location of the position of drill bit 44. As illustrated in FIG. 2B, shroud 32 may include indicators 58A, 58B, 58C, and 58D that each include a raised portion or a recessed portion of shroud 32 to provide a tactile indication of drill bit 44 relative to shroud 32. For example, an operator may touch or manipulate indicators 58A, 58B, and 58C under scalp 20 to determine a position of drill bit 44 relative to cranium 13. Indicators 58A and 58B may extend circumferentially, relative to the X-X axis, around housing 34 and may be positioned to correspond to a proximal end and a distal end of primary cutting surface 48 of drill bit 44. Indicator 58C may extend parallel to the X-X axis on housing 34, or run in a longitudinal direction, and may include a proximal end and a distal end corresponding to a proximal end and a distal end of primary cutting surface 48 of drill bit 44. In some examples, extension member 35 may include an indicator 58D. Indicator 58D may provide an operator to determine a distance of drill bit 44 relative to incision 16. Additionally, or alternatively, indicators 58A, 58B, 58C, and 58D may include L_{E}D lights visible to the operator from under the scalp. For example, shroud 32 may include wiring and an electrical coupling configured to electrically couple indicators 58A, 58B, 58C, and 58D including L_{E}D lights to a power source. In other examples, indicators 58A, 58B, 58C, and 58D may include colored or fluorescent markings. In some examples, indicators 58A, 58B, 58C, and 58D may be used in combination with templates 21 discussed above. By using indicators 58A, 58B, 58C, and 58D, an operator may more accurately determine a position of drill bit 44 when shroud 32 is under scalp 20 of patient 12.

A proximal portion of housing 34 and/or extension member 35 may include a rotation facilitating member 62 as shown in FIG. 2C. In some examples, rotation facilitating member 62 may define an axial channel configured to surround at least a portion of shank 46. In some examples, rotation facilitating member 62 may include a bearing or bushing fixed to housing 34 and/or extension member 35. The bearing or bushing may allow shank 46 to rotate and reduce clearance between shank 46 and housing 34 and/or extension member 35. By reducing clearance between shank 46 and housing 34 and/or extension member 35, rotation facilitating member 62 may reduce vibration or eccentric motion of drill bit 44 during operation of drill bit 44. In some examples, by reducing clearance between shank 46 and housing 34, rotation facilitating member 62 may improve confinement of irrigation fluid to cavity 42. Confining irrigation fluid to cavity 42 may improve removal of debris from cavity 42 and/or reduce heat accumulation in cavity 42. Additionally, or alternatively, rotation facilitating member 62 may reduce an amount of irrigation fluid backflowing through the axial channel defined by rotation facilitating member 62 toward the operator (e.g., in the anterior direction).

In some examples, shroud 32 may include an irrigation port 64. Irrigation port 64 may be coupled to or integrally formed with shroud 32. Although illustrated in FIGS. 2A and 2B as coupled to collar 36, in other examples, irrigation port 64 may be coupled to or integrally formed with housing 34 or extension member 35. Alternatively, a plurality of irrigation ports 64 may be coupled to or integrally formed with housing 34, extension member 35, and/or collar 36. Irrigation port 64 may be configured to couple to an elongated member 66 (e.g., a rigid or flexible tube) defining a lumen. Elongate member 66 may be fluidly coupled to an irrigation fluid source to supply an irrigation fluid, such as, for example, a saline solution, through the lumen to irrigation port 64 and into at least one of housing 34, extension member 35, and collar 36. Irrigation port 64 may deliver irrigation fluid to at least one of housing 34, extension member 35, and collar 36 to at least one of cool drill bit 44, shank 46, and/or rotation facilitating member 62, or flush debris from cavity 42 or extension member cavity 37. In some examples, shroud 32 may include a suction port configured to remove debris from shroud 32, such as from housing 34.

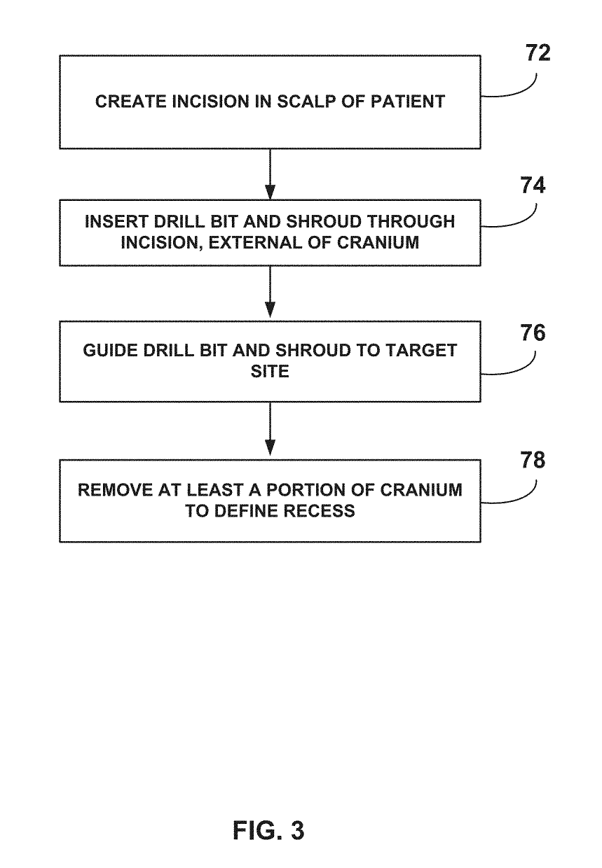

FIG. 3 is a flow diagram illustrating an example technique of creating a recess 15 in bone or substantially rigid tissue of a patient to facilitate the removal of tissue for implantation of an implantable medical device. The technique of FIG. 3 will be described with concurrent reference to medical device 10 of FIG. 1 and bone drill 30 of FIGS. 2A-2C, although the technique may be performed by another system or tool described herein or with similar tools, and that medical device 10 and bone drill 30 may be used to perform other techniques.

The technique illustrated in FIG. 3 includes creating incision 16 in scalp 20 of patient 12 (72). An operator may create using any suitable method (e.g., a scalpel) to create incision 16 in scalp 20. In some examples, creating incision 16 includes determining an orientation, shape, and size of incision to enable the operator to maneuver bone drill 30 under scalp 20 external to cranium 13.

The technique illustrated in FIG. 3 also includes inserting drill bit 44 and shroud 32 through incision 16 in scalp 20 and external of cranium 13 of patient 12 (74). As discussed above with reference to FIGS. 2A-2C, drill bit 44 and shroud 32 may be attached to bone drill 30. For example, collar 36 of shroud 32 may be coupled to drill housing 31 or bone drill 30 and shank 46 of drill bit 44 may be coupled to a coupling (e.g., chuck) at a distal end of a drive shaft of bone drill 30.

The technique illustrated in FIG. 3 also includes guiding drill bit 44 and shroud 32 under scalp 20 and to a target site (e.g., a desired location of recess 15) (76). As discussed above with reference to FIGS. 2A-2C, shroud 32 may include housing 34 extending along an axis (e.g., X-X axis) and defining inner surface 38 around the axis, inner surface 38 terminating at edge 40 of housing 34 and defining cavity 42, where housing 34 is configured to receive drill bit 44 having shank 46 and primary cutting surface 48 extending substantially parallel to the axis; and collar 36 coupled to housing 34, collar 36 configured to engage a distal portion of bone drill 30. In some examples, guiding drill bit 44 and shroud 32 may include visually or tactilely guiding shroud 32 under scalp 20. In example in which shroud 32 includes at least one indicator (e.g., one or more of indicators 58A, 58B, 58C, and/or 58D) configured to provide at least one of a tactile indication or a visual indication of a location of drill bit 44 relative to shroud 32, the technique illustrated in FIG. 3 optionally includes, aligning at least one indicator with at least a portion of the target site, as discussed above.

The technique illustrated in FIG. 3 also includes removing, by bone drill 30, at least a portion of cranium 13 to define recess 15 (78). For example, after guiding bone drill 30 to the target site, an operator may operate the motor of bone drill 30 to cause drill bit 44 to rotate and cut away at least a portion of cranium 13. In some examples, one or more portions of shroud 32, such as, for example, edge 40, flange 52, and/or other components, may be configured to contact an outer surface of cranium 13 of patient 12 to improve handling or stability of bone drill 30 as bone drill 30 is operated to form recess 15. In addition, edge 40 and/or flange 52 may enable the recess to be formed to a predetermined depth that corresponds to the distance drill bit 44 extends beyond the plane defined by flange 52. For example, in examples in which shroud 32 includes flange 52 extending radially outward from edge 40 of housing 34, removing at least a portion of the cranium may include resting surface 54 of flange 52 against an outer surface of cranium 13. In examples in which shroud 32 includes one or more irrigation ports 64, removing at least a portion of the cranium may optionally include delivering an irrigation fluid through irrigation port 64 to at least one of cool drill bit 44 or flush debris from shroud 32.

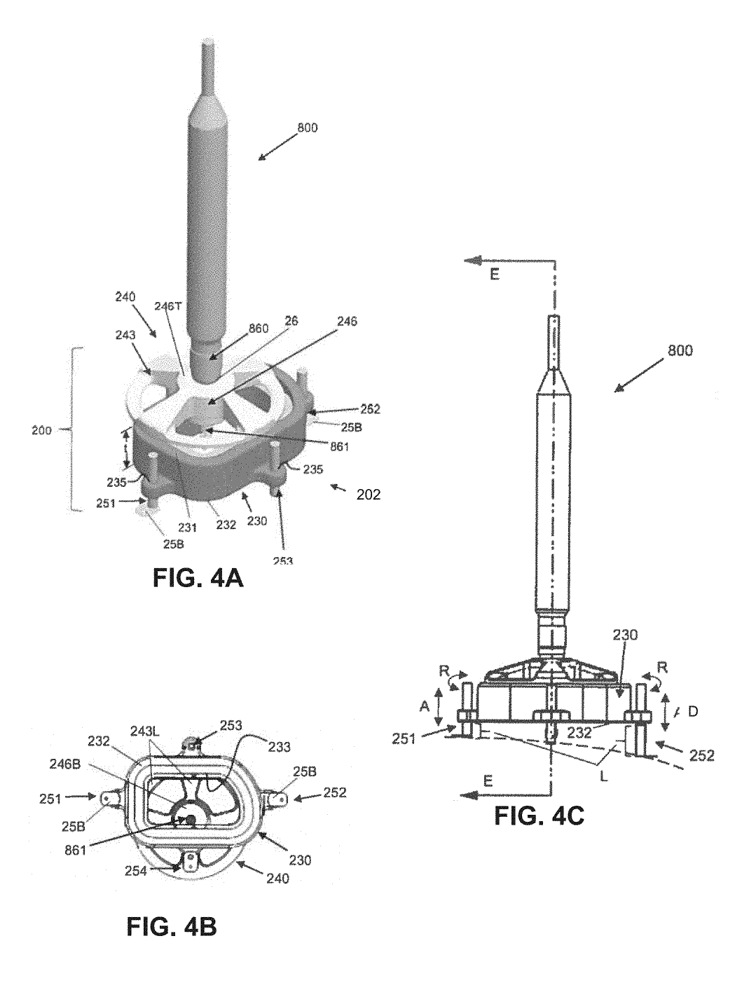

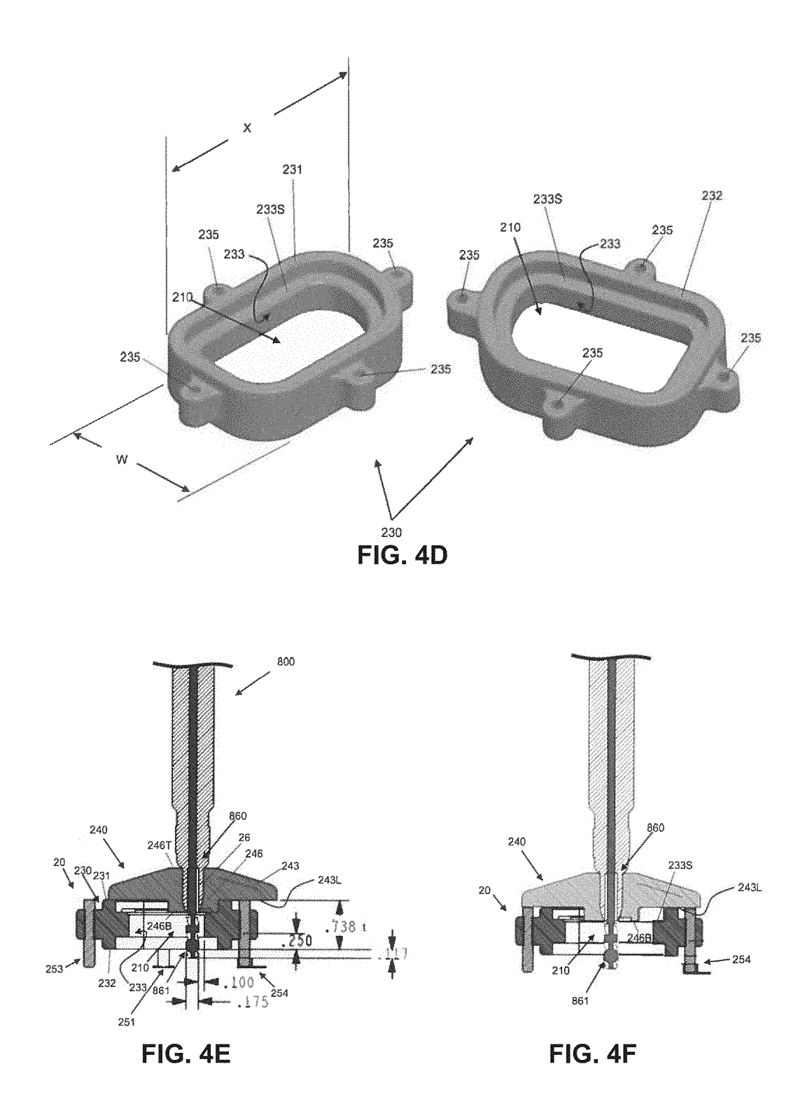

FIGS. 4A-4B are a perspective view, and a bottom plan view, respectively, of a system 200 set up to guide a tool 800 (e.g., a Medtronic Midas Rex® Surgical Drill) in forming a cranial recess (e.g., recess 15), according to some embodiments. FIGS. 4A-4B illustrates system 200 including a template assembly 202 and a shroud 240, which is attached to tool 800 to form an interface between tool 800 and a frame 230 of template assembly 202, as described in greater detail below. Template assembly 202 and shroud 240 may be formed, molded and/or or machined according to methods known in the art, from any suitable relatively rigid plastic and/or metal material known in the art.

Frame 230 of template assembly 202 is shown having a first surface 231, a second surface 232, opposite first surface 231, and third surface 233 (FIG. 4D), which extends from first surface 231 to second surface 232 and defines a perimeter of an aperture 210 (FIG. 4D) of template assembly 202. FIGS. 4A4B further illustrate template assembly 202 including a plurality of support members 251, 252, 253, and 254 coupled to frame 230, wherein a first support member 251 is coupled on a first side of aperture 210, and a second support member 252 is coupled on a second side of aperture 210, opposite the first side, and support members 253, 254 are likewise coupled on opposing sides of aperture 210. Each support member 251, 252, 253, and 254 is configured for confronting engagement with the outer surface of the cranium or the scalp adjacent to the incision, and, according to the illustrated embodiment, at least first and second support members 251, 252 include a base 25B that is configured for attachment to a cranium (e.g., cranium 13), for example, via a surgical fastener, or bone screw, received through an aperture of each, for temporary fixation of assembly 202 during the formation of the cranial recess. Template assembly aperture 210 projects onto the outer surface of the cranium, when frame 230 is attached thereto, and support members 251, 252, 253, and 254 stabilize the attached frame 230. The projection of aperture 210 defines a shape of the recess to be formed in the cranium, which corresponds to that of the IMD to be implanted therein.

With reference to the elevation view of FIG. 4C, a dashed line “D” depicts an exemplary curvature of the outer surface of the cranium. To accommodate this curvature, each of at least first and second support members 251, 252 is configured so that a length L thereof (shown extending from second surface 232 of frame 230) is independently adjustable, per arrows A. FIG. 4C illustrates second support member 252 being adjusted to a longer length than that of first support member 251. Support members 253, 254 may likewise be adjustable to account for a curvature of the cranium along another direction (into the page). According to an exemplary embodiment, a threaded coupling between a threaded exterior of each support member 251, 252, 253, and 254 and a corresponding threaded bore 235 formed in frame 230 (best seen in FIG. 4D) allows the adjustment per arrows A by rotating support members 251-254 per arrows R.

With further reference to FIGS. 4A-4C, shroud 240 includes an inner collar 246 and an outer collar 243, wherein outer collar 243 has a lower surface 243L extending between an outer perimeter thereof and inner collar 246, and inner collar 246 includes a bore 26 extending from a first opening thereof, at a top side 246T of inner collar 246, to a second opening thereof, at a bottom side 246B of inner collar 246. A shaft assembly 860 of tool 800 is shown being received within inner collar bore 26 so that a bit 861 of shaft assembly 860 protrudes from the second opening of bore 26. According to the illustrated embodiment, tool 800, with shroud 240 attached thereto, can be positioned with shroud outer collar lower surface 243L confronting first surface 231 of template assembly frame 230, so that outer collar 243 spans aperture 210 and tool bit 861 extends through aperture 210. According to some embodiments, with reference to perspective views of FIG. 4D, frame first and second surfaces 231, 232 are essentially the same for confronting and sliding engagement of shroud outer collar lower surface 243L, and the above-described threaded coupling between support members 251, 252, 253, and 254 and frame 230 allow for extension of support members 251, 252, 253, and 254 from either surface 231, 232 of frame 230. Thus, either first surface 231 of frame 230 can be selected to face toward the outer surface of the cranium, or second surface 232 can be selected to face toward the outer surface of the cranium, so that, with reference back to FIG. 1B, alternative orientations (dashed lines versus solid lines) of the shape of template assembly aperture 210 can be projected for forming recess 15.

With further reference to FIG. 4D, according to some embodiments, third surface 233 of template assembly frame 230 includes a shelf 233S formed therein, wherein an edge of shelf 233 S defines the perimeter of aperture 210; and, with reference to FIGS. 4E and 4F, when shroud outer collar lower surface 243L confronts frame first surface 231 (or second surface 232, if first surface 231 is selected to face toward the cranium) bottom side 246B of shroud inner collar 246 is substantially coplanar with the side of shelf 233S that faces toward lower surface 243L. FIGS. 4E and 4F are cross-section views, per section line E-E of FIG. 4C, that show two positions of tool 800 relative to template assembly frame 230, as the sliding engagement interface between shroud 240 and frame 230 serves to guide the movement of tool 800 in the forming of the cranial recess.

According to the illustrated embodiment, shroud inner collar 246 protrudes from shroud outer collar lower surface 243L so that bottom side 246B of inner collar 246 is spaced apart from lower surface 243L. Thus, inner collar 246 maintains a gap between tool bit 861 and frame third surface 233. FIG. 4E illustrates a minimum gap between bit 861 and third surface 233, for example, being approximately 0.1 inch, when shroud 240 is positioned so that bottom side 246B of inner collar 246 rests on shelf 233S as bit 861 is moved to form a perimeter of a cranial recess along the projected perimeter of template assembly aperture 210. FIG. 4F illustrates shroud 240 moved to a position at which inner collar 246 no longer rests on third surface shelf 233S, but outer collar lower surface 243L, with outer collar 243 spanning aperture 210, still confronts, in sliding engagement, frame 230 so that tool bit 861 can mill, or grind, the cranial recess within the perimeter thereof. With further reference to FIGS. 4A and 4E a thickness “t” of frame 230, defined from first surface 231 to second surface 232, may be substantially uniform around the perimeter of aperture 210, so that a distance between the second opening of bore 26 (at bottom side 246 B of inner collar 246) and aperture 210, in a direction of thickness “t”, remains substantially the same no matter the position of confronting lower surface 243L of shroud outer collar 243. FIG. 4E further indicates some exemplary dimensions (in inches) of a particular embodiment of frame 230, and with reference back to FIG. 4D, a corresponding length “X” of frame may be about 3 inches and a corresponding width “W” about 2 inches.

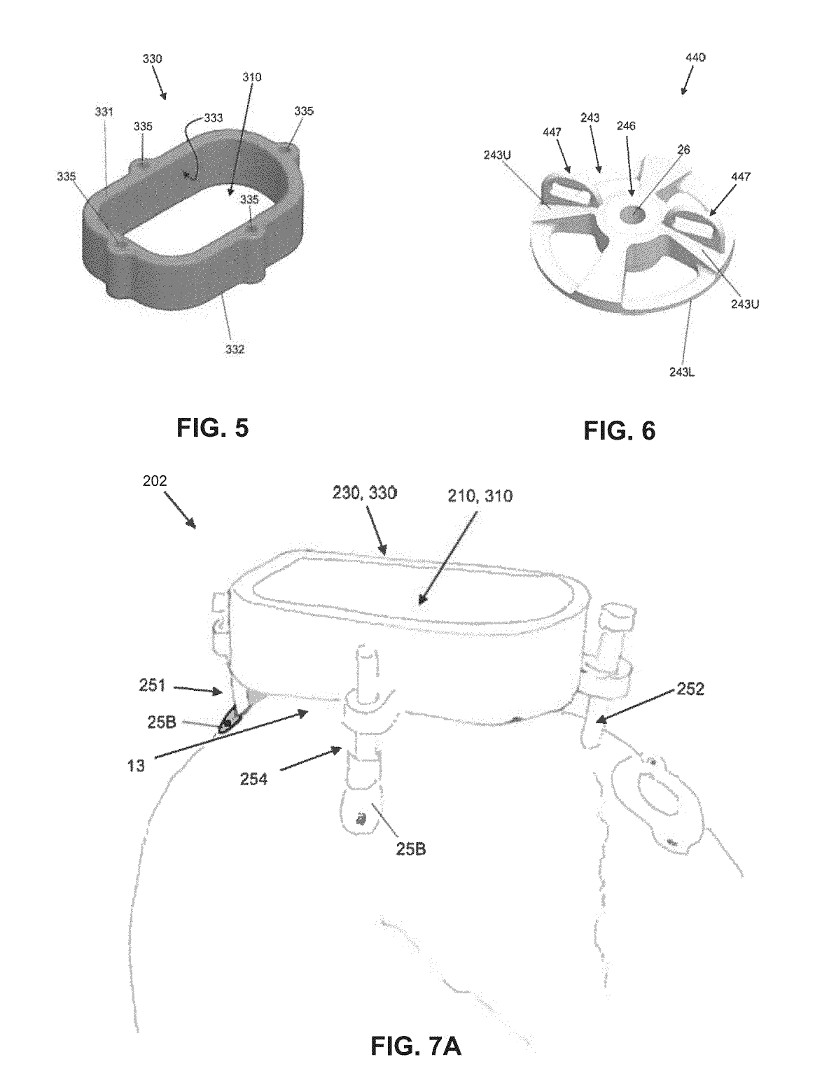

FIG. 5 is a perspective view of a template assembly frame 330, according to some alternate embodiments, which may be employed, in lieu of frame 230, by system 200. FIG. 5 illustrates frame 330 including a first surface 331, a second surface 332, opposite first surface 331, and a third surface 333 that extends from first surface 331 to second surface 332 and defines a perimeter of an aperture 310. In contrast to third surface 233 of frame 230, third surface 333 of frame 330 does not include any shelf formed therein. FIG. 5 further illustrates frame including threaded bores 335 similar to bores 235 of frame 230, each of which may provide a threaded coupling for a corresponding support member 251, 252, 253, and 254.

FIG. 6 is a perspective view of a shroud 440, according to some alternate embodiments, which may be used, in lieu of shroud 240, by system 200. FIG. 6 illustrates shroud 440, like shroud 240, including inner collar 246 and outer collar 243. But in contrast to shroud 240, shroud 440 further includes at least one handle 447 protruding from an upper surface 243U of outer collar 243, wherein handle(s) 447 may assist an operator in guiding the movement of tool 800 in forming the cranial recess, when shroud lower surface 243L is in sliding engagement with frame 230 or frame 330.

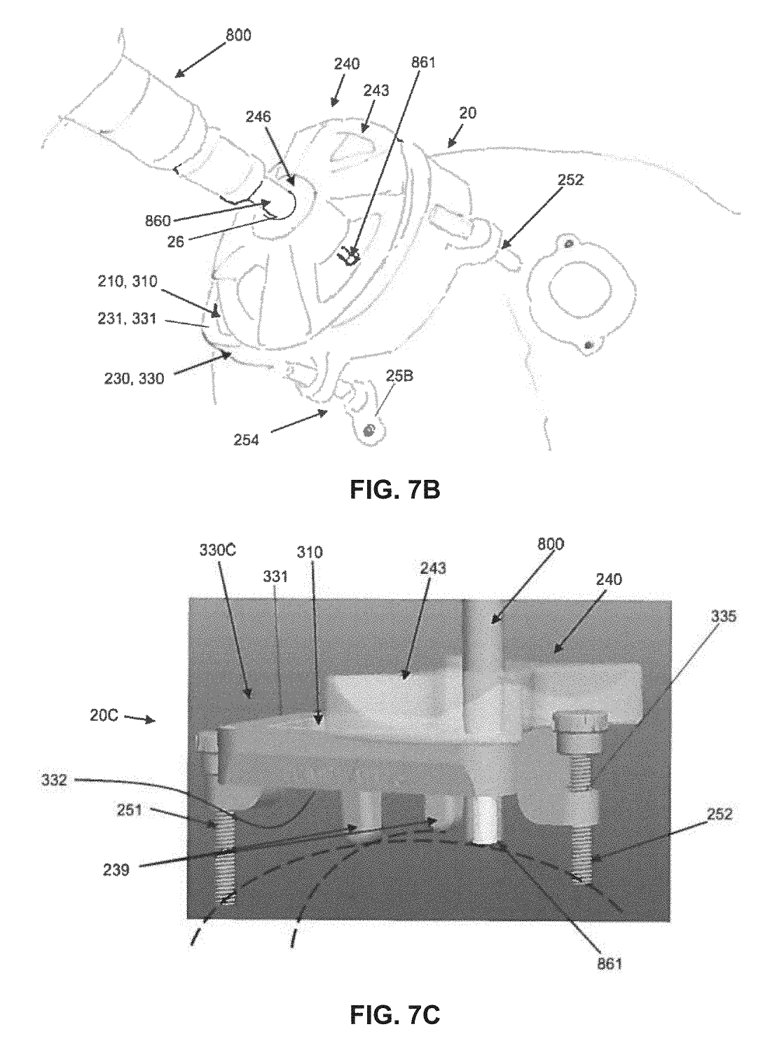

FIG. 7A and 7B are conceptual illustrations that outline an example method for forming the cranial recess after an incision through the patient's scalp, to expose an outer surface of cranium 13 has been made. FIG. 7A illustrates bases 25B of at least support members 251, 254 of template assembly 202 being attached to cranium 13, so that frame 230, 330 is secured in place with aperture 210, 310 projecting onto the outer surface of cranium 13, after an operator has independently adjusted the lengths of support members 251, 252, 253, and 254 and positioned template assembly 202 over the outer surface of cranium 13 with support members 251, 252, 253, and 254 in confronting engagement with cranium 13. It should be noted that in some instances some of support members 251, 252, 253, and 254, for example, those that do not include base 25B, like support member 252 seen in FIG. 7A, may confront the patient's scalp adjacent to the incision (not shown). FIG. 7B illustrates shroud 240, having been attached to tool 800 by inserting shaft assembly 860 thereof with bore 26 of shroud inner collar 246, and being positioned so that lower surface 243L of shroud outer collar 243 confronts first surface 231, 331 of template assembly frame 230, 330 for sliding engagement therewith. FIG. 7B further illustrates tool bit 861 extending from shroud 240 and through template assembly aperture 210, 310 for the forming of a cranial recess, for example, cranial recess 15 (FIGS. 1A and 1B), when the operator moves tool 800 by sliding the confronting shroud 240 along surface 231, 331, being constrained by third surface 233, 333 of template assembly frame 230, 330. If shroud 440 of FIG. 6 is employed in lieu of shroud 240, the operator may grasp handle(s) 447 to slide the confronting shroud along surface 231, 331.

FIG. 7C is a conceptual illustration showing an example template assembly 202C, according to an alternate embodiment, that may be employed in system 200 in lieu of assembly 202. Template assembly 202C is shown positioned over a patient's cranium that is represented with dashed lines. FIG. 7C illustrates a frame 330C of assembly 202C including only two threaded bores 335, for the threaded coupling of support members 251, 252, and two integrated fixed-length support members 239 extending from second surface 332 of frame 330C. FIG. 7C further illustrates shroud 240 positioned so that shroud outer collar 243 confronts first surface 331 of frame 330 for sliding engagement therewith, and tool bit 861 extending from shroud 240 and through template assembly aperture 310 for the forming of a cranial recess, for example, recess 15 (FIG. 1B).

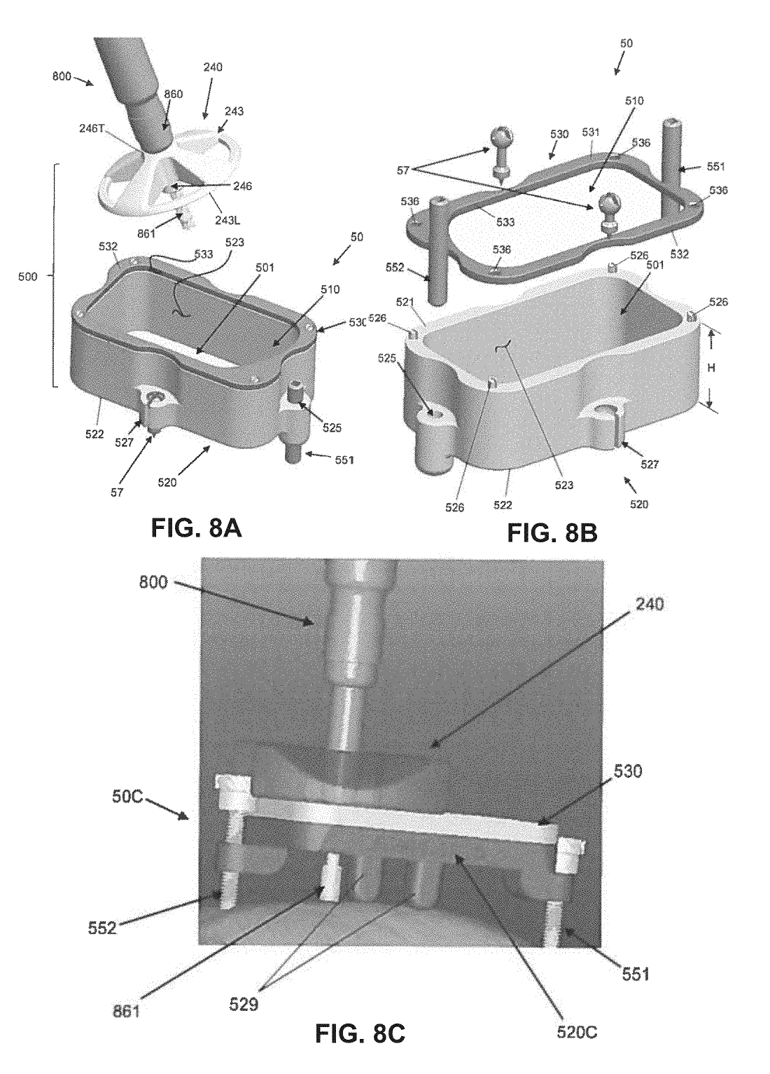

FIG. 8A is a perspective view of a system 500 according to some alternate embodiments. FIG. 8A illustrates system 500 including a template assembly 50 and the previously described shroud 240, which is attached to tool 800 to form an interface between tool 800 and template assembly 50. FIG. 8B is an exploded perspective view of template assembly 50. Template assembly 50 may be formed, molded and/or machined according to methods known in the art, from any suitable relatively rigid plastic and/or metal material known in the art. FIGS. 8A and 8B illustrate template assembly 50 including a frame 530 and a jig 520, to which frame 530 can be reversibly coupled, either in a first orientation (FIG. 8A) or a second orientation (FIG. 8B), for example, as described below. Similar to frame 230 of template assembly 202, frame 530 has a first surface 531, a second surface 532, opposite first surface 531, and third surface 533, which extends from first surface 531 to second surface 532 and defines a perimeter of an aperture 510 of template assembly 50. FIGS. 8A and 8B further illustrate jig 520 including a top surface 521, a bottom surface 522, and an inner surface 523 that extends from top surface 521 to bottom surface 523 and defines an aperture 501 of jig 520, which, when frame 530 is coupled to jig 520, is generally aligned with template assembly aperture 510 and has a footprint larger than the projection of template assembly aperture 510. In FIGS. 8A, tool 800, with shroud 240 attached thereto, is shown being directed toward assembly 50 so that shroud outer collar lower surface 243L will confront frame second surface 532 with outer collar 243 spanning apertures 501, 510 and with tool bit 861 extending through apertures 501, 510.

With further reference to FIGS. 8A and 8B, frame 530 is coupled to jig 520 by a press fit of each of a plurality of protrusions 526, formed on top surface 521 of jig 520, within a corresponding through-hole 536 formed through frame 530. According to the illustrated embodiment, either frame first surface 531 can be selected to confront jig top surface 521 (FIG. 8A) so that shroud outer collar lower surface 243L can confront frame second surface 532 in sliding engagement, or frame second surface 532 can be selected to confront jig top surface 521 (FIG. 8B) so that shroud outer collar lower surface 243 can confront frame first surface 531 in sliding engagement. Thus, depending upon the orientation of frame 530 relative to jig 520, alternative orientations of the shape of template assembly aperture 510 can be projected for forming a cranial recess, for example, recess 15 (FIG. 1B).

FIGS. 8A and 8B further illustrate template assembly 50 including first and second support members 551, 552, wherein each support member 551, 552 is configured for confronting engagement with the outer surface of the cranium or the scalp adjacent to the incision, and a length of each (extending below jig bottom surface 522) is independently adjustable, for example, by a threaded coupling, to accommodate a curvature of the patient's cranium, as described above. According to the illustrated embodiment, the threaded coupling between support members 551, 552 and jig 520 is formed by a threaded exterior of each support member 251, 252 and a corresponding threaded bore 525 formed in jig 520. Template assembly jig 520 may also, preferably, include a pair of fittings 527, each being configured to receive attachment of a corresponding fiducial 57, each of which is configured for fastening to the cranium. Fiducials 57 are best seen in FIG. 8B. Only one of fittings 527 can be seen in FIGS. 8A and 8B, but it should be understood that the other of the pair is located on an opposite side of aperture 501. A ball and socket interface between fiducials 57 and fittings 527, according to some embodiments, may provide some flexibility, or additional degrees of freedom in fastening fiducials 57.

According to some methods, fiducials 57 may be fastened to the cranium, for example, being positioned via another template (not shown), and then each fastened fiducial 57 is attached to a corresponding fitting of jig 520 to position template assembly 50 with aperture 510 projecting onto the outer surface of the cranium. After positioning template assembly 50, an operator can form a cranial recess with system 500 and tool 800, for example, in the same manner described above in conjunction with FIG. 7B, that is, with shroud outer collar 243 confronting either first surface 531 or second surface 532 of frame 530 in sliding engagement so that tool bit 861 extends through apertures 510, 501. If one or both of support members 551, 552 rest on the scalp alongside a relatively smaller incision site, it may be appreciated that the larger aperture 501 of jig 520 may partially project onto the scalp, but the smaller template assembly aperture 510 that guides tool 800 with shroud 240 attached will only project onto the outer surface of the cranium that is exposed with the incision. With further reference to FIG. 8B, a height “H” of jig 520 may be such to provide adequate clearance from the incision site for the operator of tool 800. The conceptual illustration of FIG. 8C shows an alternate embodiment template assembly 50C that includes a jig 520C having a pair of integral fixed-length support members 529, in lieu of fittings 527.

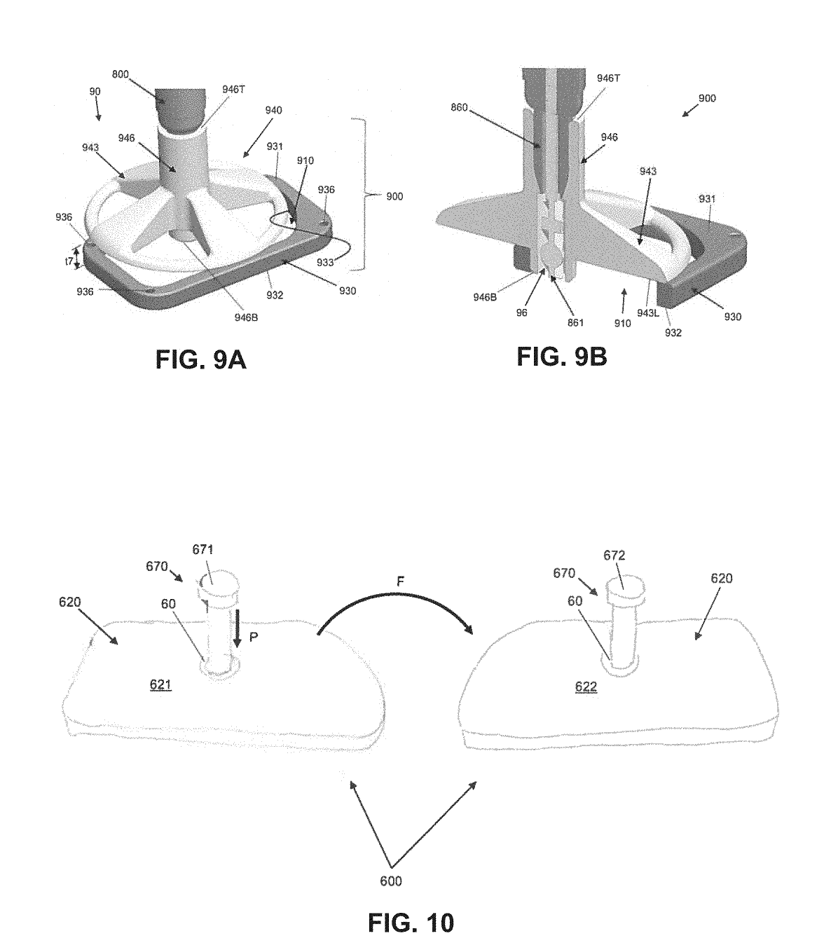

FIG. 9A is a perspective view of a system 900, according to some additional alternate embodiments; and FIG. 9B is a cross-section view through system 900. FIGS. 9A and 9B illustrate system 900 including a template frame 930 and a shroud 940, which is attached to tool 800 to form an interface between tool 800 and frame 930. Template frame 930 and shroud 940 may be formed, molded and/or or machined according to methods known in the art, from any suitable relatively rigid plastic and/or metal material known in the art.

Template frame 930 is shown having a first surface 931, a second surface 932, opposite first surface 931, and third surface 933, which extends from first surface 931 to second surface 932 and defines a perimeter of an aperture 910 of frame 930. A thickness “t7” of frame 930 is defined from first surface 931 to second surface 932, and is such to allow insertion of edges of frame 930 beneath the scalp of the patient so that a size of the incision site that exposes the cranium can be reduced. Frame 930 is shown including through-holes 936 configured to receive a surgical fastener, or bone screw therethrough for the temporary attachment of frame 930 to the cranium. FIGS. 9A and 9B illustrate shroud 940 including an outer collar 943 and an inner collar 946, which has a bore 96 extending from a first opening thereof, at a top side 946T of inner collar 946, to a second opening thereof, at a bottom side 946B of inner collar 946. Shaft assembly 860 of tool 800 is shown being received within inner collar bore 96 so that bit 861 of shaft assembly 860 protrudes from the second opening of bore 96.

FIGS. 9A and 9B further illustrate a lower surface 943L of shroud outer collar 943 confronting first surface 931 of template frame 930, so that outer collar 943 spans aperture 910 and tool bit 861 extends through aperture 910. With further reference to FIG. 9B, shroud inner collar 946 can be seen protruding from shroud outer collar lower surface 943L so that inner collar 946 maintains a gap between tool bit 861 and frame third surface 933. Similar to template assemblies 20, 50 described above, aperture 910 projects onto the outer surface of the cranium, when frame 930 is attached thereto, and the projection of aperture 910 defines a shape of the recess to be formed in the cranium, which corresponds to that of the IMD to be implanted therein. Frame first and second surfaces 931, 932 are essentially the same for confronting and sliding engagement of shroud outer collar lower surface 943L, so that either first surface 931 can be selected to face toward the outer surface of the cranium, or second surface 932 can be selected to face toward the outer surface of the cranium, and, with reference back to FIG. 1B, alternative orientations (dashed lines versus solid lines) of the shape of template frame aperture 910 can be projected for forming recess 15.

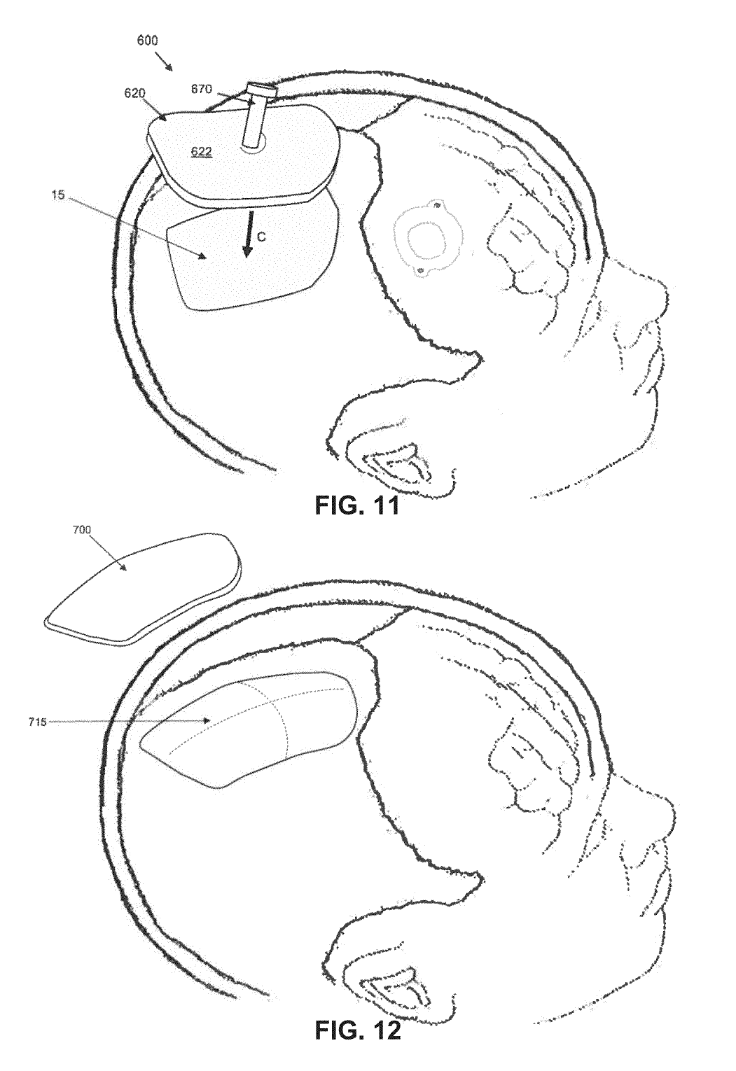

Once the cranial recess is formed, and before removing template assembly 202, 50, or template frame 930 from cranium 13, the operator may check the recess with a sizer 600, which may be included as part of template assembly 202, 50 and is shown in perspective views FIG. 10, and in the conceptual illustration of FIG. 11. (For clarity in illustration, template assembly 202, 50/frame 930 is not shown in FIG. 11.) FIGS. 10 and 11 illustrate sizer 600 including a body 620 that has a footprint corresponding to the projection of template assembly aperture 210, 310, 510, 910 and wherein the footprint and a thickness of body 620 mimics that of the IMD intended for implant in cranial recess 15. FIGS. 10 and 11 further illustrate sizer 600 including a handle 670, which is coupled to body 620 in a manner that allows the operator to adjust handle 670 so that it protrudes from either a first side 621 of body 620 or a second side 622 of body 620, depending on the orientation of the cranial recess. For example, with reference back to FIG. 1B, sizer 600 with handle 670 protruding from body first side 621 corresponds to the dashed line representation of the cranial recess, while sizer 600 with handle 670 protruding from body second side 622 corresponds to the solid line representation of the cranial recess. According to the illustrated embodiment, handle 670 extends within a through-bore of body 620, being retained therein by enlarged opposing ends 671, 672 thereof, so that, according to some methods, the operator can move handle 670, per arrow P, from first side 621 to second side 622. (In FIG. 10, when sizer 600 of the left-hand side perspective view is flipped, per arrow “F”, and handle 670 is moved, per arrow “P”, the right-hand perspective view is the result.) FIG. 11 illustrates sizer 600, with handle 670 protruding from second side 622, so that the operator can grasp handle 670 to position sizer 600 as shown, and then insert sizer 600 into cranial recess 15, per arrow “C”, to make sure that the perimeter and depth thereof will be suitable for implanting the IMD therein. In other examples, the operator may grasp sizer 600 with a surgical tool, such as, for example, by grasping slots integrally formed in sizer 600 with a hemostat, or a handle tethered to sizer 600.

With reference to the conceptual illustration of FIG. 12, for cases when an IMD 700 has a curved contour across a thickness thereof, to match the contour of the outer surface of the patient's cranium, some alternate embodiments of system 200 can have features to facilitate the formation of a cranial recess 715 that has a convex floor, also matching the contour of the outer surface of the cranium. For example, in alternate embodiments of system 200, one or both of first and second surfaces 231, 331, 232, 332 of frame 230, 330 may have a curved contour to facilitate the milling/grinding of cranial recess 715.

The foregoing detailed description, in conjunction with appended drawings, is exemplary in nature and is not intended to limit the scope, applicability, or configuration of inventive embodiments disclosed herein in any way. Rather, the foregoing description provides practical examples, and those skilled in the art will recognize that some of the examples may have suitable alternatives. In the drawings, which are not necessarily to scale, like numerals/letters denote like elements. Examples of constructions, materials, dimensions and fabrication processes are provided for select elements and all other elements employ that which is known by those skilled in the art. It is appreciated that various modifications and changes can be made without departing from the scope of the disclosure as set forth in the appended claims, and various combinations of elements described above in conjunction with the specific embodiments, are within the scope of the present claims. It should be noted that the systems described herein may not be limited to treatment of a human patient. In alternative examples, these systems may be implemented in non-human patients, e.g., primates, canines, equines, ovines, pigs, and felines. These animals may undergo clinical or research therapies that my benefit from the subject matter of this disclosure.

The following examples illustrate subject matter of the present disclosure.

Example 1. A shroud for a bone drill, the shroud comprising: a housing extending along an axis and defining an inner surface around the axis, the inner surface terminating at an edge of the housing and defining a cavity, wherein the cavity is configured to receive a drill bit having a shank and a primary cutting surface extending substantially parallel to the axis; and a collar coupled to the housing, the collar configured to engage a distal portion of the bone drill.

Example 2. The shroud of Example 1, wherein the housing is configured to enable the primary cutting surface of the drill bit to radially extend between approximately 2 millimeters to approximately 4 millimeters beyond the edge of the housing.

Example 3. The shroud of any of Examples 1 or 2, comprising a flange extending radially outward from the edge of the housing, wherein a surface of the flange is configured to contact an outer surface of a cranium of a patient.

Example 4. The shroud of any of Examples 1 through 3, wherein a length of the housing in the axial direction is between approximately 3 centimeters and approximately 5 centimeters.

Example 5. The shroud of any of Examples 1 through 4, further comprising an extension member coupling the housing to the collar.

Example 6. The shroud of Example 5, wherein a length of the extension member in the axial direction is approximately equal to a length of the housing in the axial direction.

Example 7. The shroud of any of Examples 1 through 6, wherein the housing defines at least one aperture through a portion of the inner surface, wherein the at least one aperture is configured to enable debris to exit the housing.

Example 8. The shroud of any of Examples 1 through 7, wherein the shroud comprises at least one indicator configured to provide at least one of a tactile indication or a visual indication of a location of the drill bit relative to the shroud.

Example 9. The shroud of Example 8, wherein the at least one indicator comprises at least one of a raised portion or a recessed portion of an outer surface of the housing, wherein the raised portion or the recessed portion extends at least one of parallel to the axis or circumferentially around the axis.

Example 10. The shroud of any of Examples 1 through 9, wherein the housing extends from a distal end to a proximal end, wherein the housing comprises: a proximal portion and a distal portion; and a rotation facilitating member defining an axial channel and positioned at the proximal portion, the rotation facilitating member configured to surround at least a portion of the shank.

Example 11. A system comprising: a drill bit having a primary cutting surface extending substantially parallel to an axis and a shank; a shroud comprising: a housing extending along the axis and defining an inner surface around the axis, the inner surface terminating at an edge of the housing and defining a cavity, wherein the cavity is configured to receive the drill bit; and a collar coupled to the housing, the collar configured to engage a distal portion of a bone drill.

Example 12. The system of Example 11, comprising a bone drill having a drill housing surrounding a motor, wherein the motor is operatively coupled to a drive shaft, wherein a distal portion of the drill housing is configured to engage the collar, wherein a distal portion of the drive shaft comprises a coupling to engage the drill bit.

Example 13. The system of any of Examples 11 or 12, wherein the housing is configured to enable the primary cutting surface of the drill bit to radially extend between approximately 2 millimeters to approximately 4 millimeters beyond the edge of the housing.

Example 14. The system of any of Examples 11 through 13, comprising a flange extending radially outward from the edge of the housing, wherein a surface of the flange is configured to contact an outer surface of a cranium of a patient.

Example 15. The system of any of Examples 11 through 14, further comprising an extension member coupling the housing to the collar.

Example 16. A method comprising: creating an incision in a scalp of a patient; inserting a drill bit and a shroud through the incision in the scalp and external of a cranium of the patient, the drill bit and the shroud attached to a bone drill; guiding the drill bit and the shroud under the scalp and to a target site, wherein the shroud comprising: a housing extending along an axis and defining an inner surface around the axis, the inner surface terminating at an edge of the housing and defining a cavity, wherein the housing is configured to receive a drill bit having a shank and a primary cutting surface extending substantially parallel to the axis; and a collar coupled to the housing, the collar configured to engage a distal portion of the bone drill; and removing, via operation of the bone drill, at least a portion of the cranium to define a recess.

Example 17. The method of Example 16, wherein the recess extends between approximately 2 millimeters to approximately 4 millimeters into the cranium.

Example 18. The method of any of Examples 16 or 17, wherein the shroud comprises a flange extending radially outward from the edge of the housing, wherein removing at least a portion of the cranium comprises resting a surface of the flange against an outer surface of the cranium.

Example 19. The method of any of Examples 16 through 18, wherein the shroud comprises at least one indicator configured to provide at least one of a tactile indication or a visual indication of a location of the drill bit relative to the shroud, wherein guiding the drill bit and the shroud comprises aligning the at least one indicator with at least a portion of the target site.

Example 20. The method of any of Examples 16 through 19, wherein the shroud comprises further comprising an irrigation port, wherein removing at least a portion of the cranium comprises delivering a fluid through the irrigation port to at least one of cool the drill bit or flush debris from the shroud.

Various examples have been described. These and other examples are within the scope of the following claims.

What is claimed is:

1. A shroud for a bone drill, the shroud comprising:
a housing extending along an axis and defining an inner surface around the axis, the inner surface terminating at an edge of the housing and defining a cavity, wherein the cavity is configured to receive a drill bit having a shank and a primary cutting surface extending substantially parallel to the axis; and
a collar coupled to the housing, the collar configured to engage a distal portion of the bone drill.

2. The shroud of claim 1, wherein the housing is configured to enable the primary cutting surface of the drill bit to radially extend between approximately 2 millimeters to approximately 4 millimeters beyond the edge of the housing.

3. The shroud of claim 1, further comprising a flange extending radially outward from the edge of the housing, wherein a surface of the flange is configured to contact an outer surface of a cranium of a patient.

4. The shroud of claim 1, wherein a length of the housing in the axial direction is between approximately 3 centimeters and approximately 5 centimeters.

5. The shroud of claim 1, further comprising an extension member coupling the housing to the collar.

6. The shroud of claim 5, wherein a length of the extension member in the axial direction is approximately equal to a length of the housing in the axial direction.

7. The shroud of claim 1, wherein the housing defines at least one aperture through a portion of the inner surface, and wherein the at least one aperture is configured to enable debris to exit the housing.

8. The shroud of claim 1, wherein the shroud comprises at least one indicator configured to provide at least one of a tactile indication or a visual indication of a location of the drill bit relative to the shroud.

9. The shroud of claim 8, wherein the at least one indicator comprises at least one of a raised portion or a recessed portion of an outer surface of the housing, and wherein the raised portion or the recessed portion extends at least one of parallel to the axis or circumferentially around the axis.

10. The shroud of claim 1, wherein the housing extends from a distal end to a proximal end, and wherein the housing comprises:
a proximal portion and a distal portion; and
a rotation facilitating member defining an axial channel and positioned at the proximal portion, the rotation facilitating member configured to surround at least a portion of the shank.

11. A system comprising:
a drill bit having a primary cutting surface extending substantially parallel to an axis and a shank;
a shroud comprising:
a housing extending along the axis and defining an inner surface around the axis, the inner surface terminating at an edge of the housing and defining a cavity, wherein the cavity is configured to receive the drill bit; and
a collar coupled to the housing, the collar configured to engage a distal portion of a bone drill.

12. The system of claim 11, comprising a bone drill having a drill housing surrounding a motor, wherein the motor is operatively coupled to a drive shaft, wherein a distal portion of the drill housing is configured to engage the collar, and wherein a distal portion of the drive shaft comprises a coupling to engage the drill bit.

13. The system of claim 11, wherein the housing is configured to enable the primary cutting surface of the drill bit to radially extend between approximately 2 millimeters to approximately 4 millimeters beyond the edge of the housing.

14. The system of claim 11, wherein the shroud comprises a flange extending radially outward from the edge of the housing, and wherein a surface of the flange is configured to contact an outer surface of a cranium of a patient.

15. The system of claim 11, wherein the shroud comprises an extension member coupling the housing to the collar.

16. A method comprising:
creating an incision in a scalp of a patient;
inserting a drill bit and a shroud through the incision in the scalp and external of a cranium of the patient, the drill bit and the shroud attached to a bone drill;
guiding the drill bit and the shroud under the scalp and to a target site, wherein the shroud comprising:
a housing extending along an axis and defining an inner surface around the axis, the inner surface terminating at an edge of the housing and defining a cavity, wherein the housing is configured to receive a drill bit having a shank and a primary cutting surface extending substantially parallel to the axis; and
a collar coupled to the housing, the collar configured to engage a distal portion of the bone drill; and
removing, via operation of the bone drill, at least a portion of the cranium to define a recess.

17. The method of claim 16, wherein the recess extends between approximately 2 millimeters to approximately 4 millimeters into the cranium.

18. The method of claim 16, wherein the shroud comprises a flange extending radially outward from the edge of the housing, and wherein removing at least a portion of the cranium comprises resting a surface of the flange against an outer surface of the cranium.

19. The method of claim 16, wherein the shroud comprises at least one indicator configured to provide at least one of a tactile indication or a visual indication of a location of the drill bit relative to the shroud, and wherein guiding the drill bit and the shroud comprises aligning the at least one indicator with at least a portion of the target site.

20. The method of claim 16, wherein the shroud comprises further comprising an irrigation port, and wherein removing at least a portion of the cranium comprises delivering a fluid through the irrigation port to at least one of cool the drill bit or flush debris from the shroud.
